(19) United States
(12) Patent Application Publication
(10) Pub. No.: US 2006/0039835 A1
(43) Pub. Date: Feb. 23, 2006

(54) Methods and devices for aromatherapy

(75) Inventors: John Nottingham, Bratenahl, OH (US); John Spirk, Gates Mills, OH (US); Douglas Whitner, Medina, OH (US); Marc Vitantonio, South Russell, OH (US); John William Nottingham, Bratenahl, OH (US); Carolyn Marie McNeeley, Brunswick, OH (US); Sarah Nottingham Antonetti, Cleveland Heights, OH (US); Lindsey Tufts JR., Eastlake, OH (US); Daniel Wanhainen, Macedonia, OH (US)

Correspondence Address: CALFEE HALTER & GRISWOLD, LLP, 800 SUPERIOR AVENUE, SUITE 1400, CLEVELAND, OH, 44114, US

(73) Assignee: Consumer Innovation Partners, LP, Cleveland, OH (US)

(21) Appl. No.: 11/206,569

(22) Filed: Aug. 18, 2005

Related U.S. Application Data
(60) Provisional application No. 60/602,801, filed on Aug. 18, 2004.

(51) Int. Cl. A61L 9/12

(52) U.S. Cl. 422/124

(57) ABSTRACT

According to one embodiment of the present invention, a scent dispenser is provided that includes a fan and a scent cartridge, the scent dispenser dispensing scent when a flow of air is directed over a fragrant or aromatic substance in the scent cartridge.

### PRIORITY CLAIM

This application claims priority to U.S. Provisional Patent Application 60/602,801, filed Aug. 18, 2004, which is incorporated herein by reference, in its entirety.

### TECHNICAL FIELD

The field of the present invention relates generally to devices and methods for dispensing scent. More particularly, the present invention relates to devices and methods for dispensing scent in vapor form without application of direct heat. In some embodiments, the present invention relates to replenishable scent dispensing devices that are configured to cooperate with plumbing fixtures so as to drive release of scent without the use of electricity, wherein the dispensing devices include scent cartridges that can be easily removed and replaced.

### BACKGROUND OF THE INVENTION

Products for generating or emanating scent are commonly known in the art. Such products are typically provided in a solid or liquid form for aerosol dispersion in the air, or in fluids, or for evaporation. Some scent products are provided in the form of devices that generate or emanate scent upon the application of heat with an electric or candle heat source.

Lacking in the art are devices and methods for dispersing scent without the requirement of applied heat, aerosolization, or dispersion in a fluid. Also lacking are devices that in some embodiments do not require electrical power. Such devices would be particularly useful in environments such as in saunas, jacuzzis, and bathing areas that include one or more of a shower, a tub, and a sink. Accordingly, it would be desirable to provide methods and devices for dispensing scent without the requirement for a heating source. It would likewise be desirable to provide devices that do not require electrical power to drive delivery of scent into the environment.

### SUMMARY OF THE INVENTION

Accordingly, the present invention provides devices and methods for dispensing scent into an environment, wherein the methods and devices do not require direct application of heat to drive dispersion of scent. In particular, the present invention provides devices in the form of replenishable dispensers for dispensing a scent into a surrounding area without the use of directly applied heat to liberate scent. The present invention also provides methods for dispensing a scent into a surrounding area without the use of directly applied heat to liberate scent.

Devices according to the present invention include a rotor having a plurality of vanes arrayed radially, in a substantially circular pattern, around a central point with a central axis defined therethrough. Rotation around the central axis of the rotor is driven by the application of mechanical force to either the rotor or to the vanes. In some embodiments, the rotational force is provided by a motor that drives rotation of at least one axle at the center of the rotor. In other embodiments, the rotational force is provided by application of mechanical force to the plurality of blades or vanes, such as by the application of a stream of water or air to the blades or vanes.

The devices also includes a scent cartridge having a an interior space for retaining an aromatic or fragrant substance and plurality of holes defined through the cartridge sufficient to allow circulation of the air through the a scent cartridge. When the scent dispenser is in use, and assembled with the scent cartridge inserted into the rotor, and the rotor is driven to rotate about its central axis, the dispenser is capable of drawing air through the openings in the scent cartridge and passing the air out through the plurality of vanes on the rotor.

Additional features and advantages of the invention will be set forth in part in the description which follows, and in part will be obvious from the description, or may be learned by practice of the invention. The features and advantages of the invention will be realized and attained by means of the elements and combinations particularly pointed out in the appended claims. It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention, and together with the description, serve to explain the principles of the invention.

### BRIEF DESCRIPTION OF THE DRAWINGS

FIG. 1 panel A shows an exploded perspective view of a first embodiment of a scent dispensing device according to the present invention; panel B shows various vane configurations;

FIG. 2 shows an exploded perspective view of components of the scent dispensing device according depicted in FIG. 1;

FIG. 3 panel A shows an exploded perspective view and panel B Shows a perspective view of one embodiment of the scent cartridges according depicted in FIG. 1; panels C and D show perspective views of flaps on one embodiment of the scent cartridges;

FIG. 4 shows an exploded perspective view of a second embodiment of a scent dispensing device according to the present invention;

FIG. 5 panel A shows an exploded perspective view of a third embodiment of a scent dispensing device according to the present invention; panels B and C show cutaway side views of the flow control feature according to some embodiments of the instant invention with flow over the turbine (B) and through a bypass channel (C);

FIG. 6 shows a side view of another embodiment of a scent dispensing device according to the present invention arranged in series with a water supply at one end, a fluid delivery fixture at the other end, and the scent dispensing device disposed therebetween; panel A shows the assembled scent dispensing device; panel B shows an opposing side view of the scent dispensing device; panel C shows the scent dispensing device in a partially disassembled state; and

FIG. 7 shows a side view of yet another embodiment of a scent dispensing device according to the present invention arranged in series with a water supply; panel A shows the assembled scent dispensing device; panel B shows the scent dispensing device in a partially disassembled state.

### DETAILED DESCRIPTION OF THE INVENTION

The present invention will now be described with occasional reference to specific embodiments of the invention. This invention may, however, be embodied in different forms and should not be construed as limited to the embodiments set forth herein. Rather, these embodiments are provided so that this disclosure will fully convey the scope of the invention to those skilled in the art.

Except as otherwise specifically defined herein, all terms used herein have the same meaning as commonly understood by one of ordinary skill in the art to which this invention belongs. The terminology used in the description of the invention herein is for describing particular embodiments only, and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities, properties, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values to the extent that such are set forth in the specific examples are reported as precisely as possible. Any numerical values, however, inherently contain certain errors necessarily resulting from error found in their respective measurements.

Except as otherwise indicated, the disclosure of all patents, patent applications (and any patents which issue thereon, as well as any corresponding published foreign patent applications), and publications mentioned throughout this description are hereby incorporated by reference herein. It is expressly not admitted, however, that any of the documents incorporated by reference herein teach or disclose the present invention.

The present invention relates to scent dispensers for dispensing scent into a surrounding area and, in particular, to scent dispensers that include a fan or rotor and a scent cartridge for delivery of scent using air flow. The invention also relates to methods for dispensing scent using air flow. The invention also relates to articles of manufacture, including kits, with one or more components of the dispensers, instructions for use of the dispensers and/or their components, or combinations thereof. The scent dispensers operate without the requirement of direct application of heat to liberate scent from a fragrant or aromatic substance. The invention is applicable to scent dispensers of varying constructions and dimensions, and to dispensers comprising combinations of multiple fans and scent cartridges.

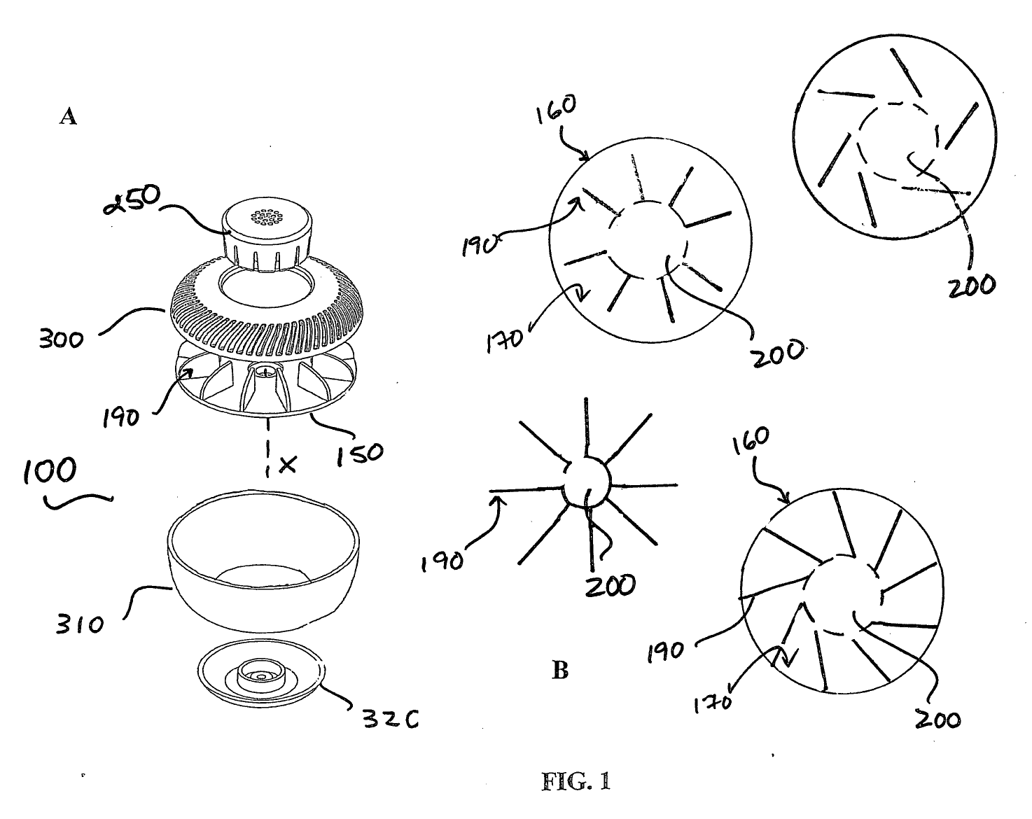

As representative of the invention, FIG. 1, panel A, illustrates a scent dispenser 100 in accordance with one embodiment of the invention. As shown in FIG. 1, the scent dispenser 100 of the illustrated embodiment generally includes at least one fan or rotor 150 having a central axis “X”, several vanes 190, and a scent cartridge 250. The depicted embodiment also includes a vented upper housing 300, a lower housing 310, and a knob 320 for actuating the scent dispenser 100 between on and off positions (detail not shown). Panel B shows several examples of vane orientations and lengths relative to the central axis X of the rotor 150 and its periphery.

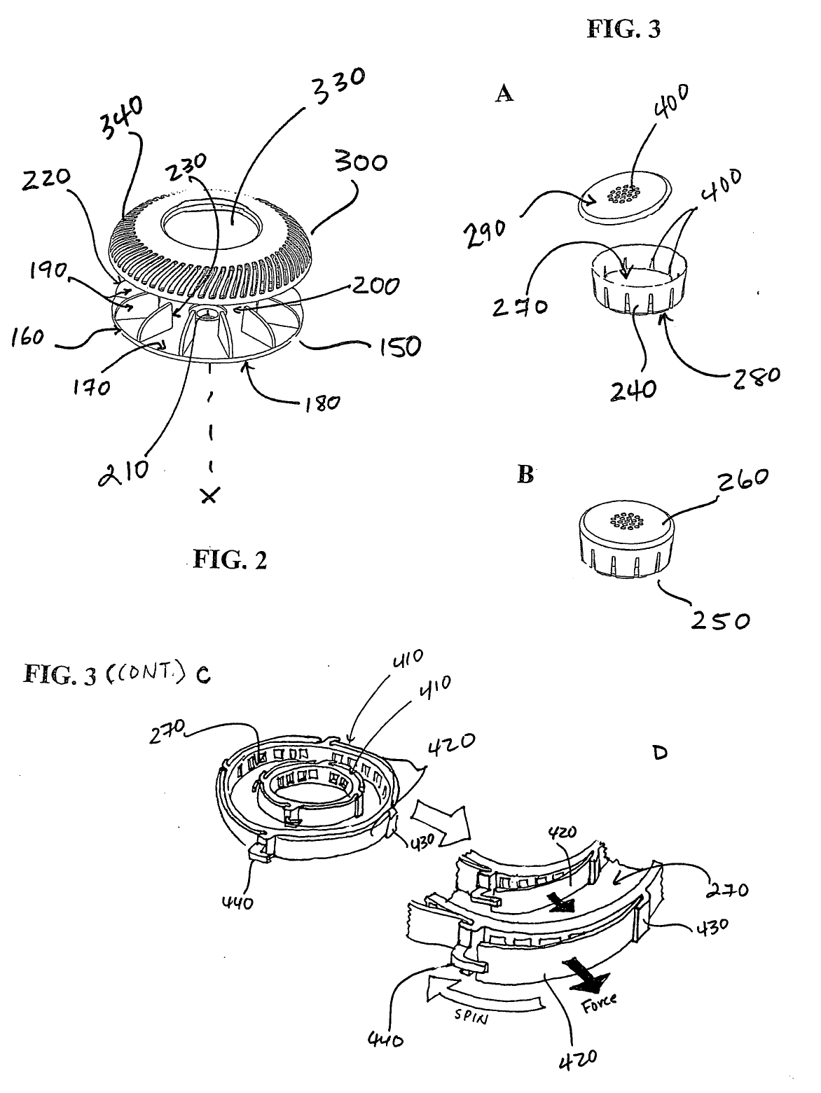

Referring now to FIG. 2, the rotor 150 has a peripheral edge 160 and substantially planar first 170 and second faces 180, at least one face 170 or 180 of the rotor 150 comprising a plurality of vanes 190. As used herein, the terms “rotor” and “fan” are used herein to refer to an assembly consisting of several blades or vanes attached to a hub or a plate, the hub or plate having a central axis around which the assembly can be rotated. The rotor 150 also has a cartridge-receiving region 200 at its center that is adapted to receive a scent cartridge 250. The depicted embodiment of the scent dispenser 100 also includes at least one scent cartridge 250 adapted for insertion into the cartridge-receiving region 200 of the rotor 150. In some embodiments, the rotor 150 has an aperture 210 through its center axis X. According to some such embodiments, the aperture 210 may serve as the cartridge-receiving region 200 for receiving the scent cartridge 250 (not shown). According to alternate forms of such embodiments, the aperture 210 operates to receive additional portions of the scent dispenser 100, such as a shaft or axle (not depicted) that functions to support the rotor 150 for rotation about its central axis X. In yet other embodiments, the rotor 250 does not have an aperture 210 at its center.

When the scent dispenser 100 is in use, and assembled with the scent cartridge 250 inserted into the rotor 150, and the rotor 150 is driven to rotate about its central axis X, the dispenser is capable of drawing air through openings in the scent cartridge 250 and passing the air out through a plurality of vanes (as described further herein). The air flow is achieved by creation of a pressure differential between the central portion of the rotor 150 and its peripheral edge 160, such that when the rotor 150 is rotating, air is drawn into the center of the rotor 150, where the relative pressure is the lowest, and is flows radially outward between the vanes 190, toward the region of relatively higher pressure at the peripheral edge 160 of the rotor 150. Of course, in alternate embodiments, such as when there is an aperture 210 at the center of the rotor 150, all or a portion of the air drawn toward the center of the rotor 150 may pass through the aperture 210, and will flow therefrom to be emitted from the scent dispenser 100.

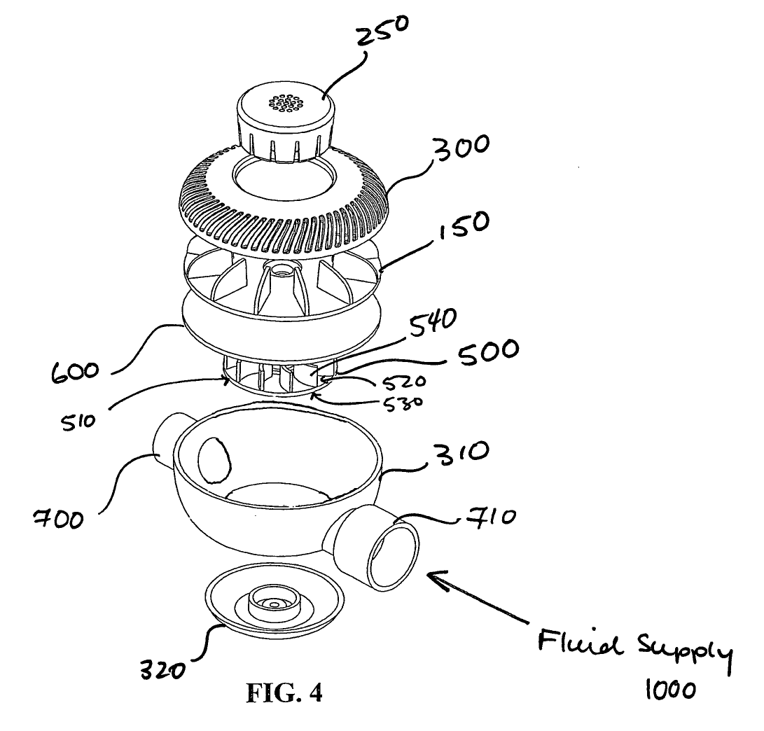

Referring still to FIG. 2, the vanes 190 are radially oriented, and each vane 190 projects from the at least one face 170 or 180 and has a length that extends from between the peripheral edge 160 of the rotor 150 toward its central axis X. As used herein, the term “vane” means a blade or paddle that is part of a set of two or more mounted in a substantially symmetrical pattern. The number of vanes 190 on a rotor 150 is 3, 4, 5, 6, 7, 8, 9 10, 11, 12, 13, 14, 15, 20, 25, 30, 35, 40, 45, or more. The depicted embodiment has approximately 14 vanes 190. In some embodiments, the vanes 190 are perpendicular relative to the face 170 or 180 of the rotor 150, as shown in the embodiment depicted in FIG. 1. In alternate embodiments, the vanes 190 are canted, in that they deviate from the vertical and the horizontal, relative to the face 170 or 180 of the rotor 150 (not shown). In some embodiments, the vanes 190 are flat and have either a rectangular profile, or have a curved edge 220, as shown in the embodiment depicted in FIG. 1. In alternate embodiments, the vanes 190 are cambered, having a generally arched shape, much like the vanes depicted in the embodiment shown in FIG. 4 herein, and corresponding to the vanes 540 of the turbine 500. The vanes 190 have a length that extends from between the peripheral edge 160 of the rotor 150 and has an end point 230 between the peripheral edge 160 of the rotor and its center axis X; thus, a portion of the rotor face 170 or 180 lacks vane 190 structure from the end point 230 of each vane 190 to the center axis X. As shown in the embodiment of the scent dispenser 100 depicted in FIG. 4, the cartridge-receiving region 200 for the scent cartridge 250 is formed by the converging end points 230 of the vanes 190, the cartridge-receiving region 200 having a generally circular shape.

The scent dispenser 100 includes a housing. As illustrated in FIG. 1, the housing of the depicted embodiment includes a cover 300 and a base 310. Both the cover 300 and base 310 are molded from plastic but other suitable materials can be used. The configurations of the cover 300 and base 310 are adapted to support and enclose other components of the scent dispenser 100. In the illustrated embodiment, both the cover 300 and base 310 have a generally dome shape, and the cover 300 is configured with an aperture 330 for insertion of the scent cartridge into the cartridge-receiving region 200 of the rotor. In various embodiments, all or a portion of the housing is vented; the depicted embodiment shows venting 340 around the periphery of the cover 300. According to embodiments having more than one rotor 150, additional portions of the housing may be vented to permit flow of air out of the housing to disperse scent when the scent dispenser 100 is in operation. In some embodiments, an actuator knob 320 is operably engaged with the components of the scent dispenser 100 internal to the housing. As depicted in FIG. 1, the actuator knob 320 is adjacent to the housing base 310, however other configurations are possible and within the skill of the art.

According to the depicted embodiment, the cover 300 and base 310 are complimentary with each other. Thus, when the cover 300 and base 310 are assembled together, they combine to form one closed space that encloses other components of the scent dispenser 100. It should be apparent to one skilled in the art, however, that the housing could be configured differently. The housing could be shaped in many different ways, for example it could be a cylindrical shape or a general box-like shape. Also, the housing could be made up of several separate parts or it could be as one piece out of a suitable material, such as molded from plastic. According to embodiments where the housing is formed of two or more pieces, the pieces, such as the cover 300 and base 310, are secured to each other by inter-engaging tabs and slots (not shown). However, several other methods of assembling housing pieces, such as the cover 300 and base 310, could be used, including techniques and fasteners including snap fitting, glue, sonic welding, screws, pegs, mated threads, etc.

Referring to FIG. 3, panels A and B, the scent cartridge 250 has an exterior surface 260, an interior space 270 for retaining an aromatic or fragrant substance (not shown), and a plurality of openings 400 through the exterior surface 260. In various embodiments, the openings 400 are generally elliptical, circular, grid, slit or slot shaped, and operate to allow the passage of air between the exterior 260 of the scent cartridge 250 and its interior space 270.

The scent cartridge 250 is generally disc-shaped, oval, spherical, hemispherical, parallelepiped-shaped, rectangular, cube-shaped, cuboid-shaped, cylindrical-shaped, pyramid-shaped, spherical-shaped, or irregularly-shaped, the plurality of openings being generally elliptical, circular, grid, slit or slot shaped. The figures generally depict a scent cartridge 250 having a disc shape. Of course it will be obvious to one of ordinary skill that the shape of the scent cartridge 250 can vary widely, the shape selected influencing the shape and configuration of the rotor cartridge-receiving region 200 into which the scent cartridge 250 is inserted. In some embodiments, the dispenser 100 is replenishable, with the scent cartridge 250 being removable and replaceable. In other embodiments, the scent cartridge 250 is permanently fixed in the rotor cartridge-receiving region 200. Referring to FIG. 3, the depicted embodiment of the scent cartridge 250 has a generally disc-shape, and has a rotor insertion face 280, an external face 290, and a substantially perpendicular edge 240 that is positioned therebetween. The rotor insertion face 280 is contacted with the rotor 150 in the rotor cartridge-receiving region 200 when the scent dispenser 100 is assembled.

The scent cartridge 250 has a plurality of openings 400. In some embodiments, the openings 400 are disposed on the external face 290 and radially around the perpendicular edge 240, as shown in FIG. 3. In alternate embodiments, the openings 400 are distributed over the exterior surface 260 of the scent cartridge 250. According to the various embodiments of the scent dispenser 100, the openings 400 in the scent cartridge 250 serve to permit air to be drawn into the interior space 270 of the scent cartridge 250 to pass over the aromatic or fragrant substance therein, and pass out of the scent cartridge 250 to be dispersed from the rotor 150. Accordingly, any arrangement of openings 400 in the scent cartridge 250 that permit such air flow are encompassed within the scope of the invention.

In some embodiments, the scent cartridges 250 according to the invention include materials that enhance the thermal conductivity of the scent cartridge 250, thus influencing the liberation of scent from the scent dispenser 100. Such thermally conductive materials and the use thereof are well known in the art, and include a variety of metals in sheet, ingot or wire form, for example; glass and polymeric materials; and other materials having thermal conductive properties. In some embodiments, the scent cartridges 250 have implements for sealing the openings 400 when a scent cartridge 250 is not in use, so as to preserve the fragrant or aromatic material contained within. The implements may be in the form of sealing films or foils that are removed prior to installation of a scent cartridge 250. In some embodiments, the film or foils are left intact on the scent cartridge 250 when it is installed in the rotor 150. One embodiment of sealing implements is shown in FIG. 3, panels C and D. The implements may be made of film or foil to form flaps 420 that are actuated to lift away from the openings 400 by the action of the air flow over and through the scent cartridge 250, or as a result of the mechanical forces (centrifugal) on the flaps 420 when the rotor 150 is in motion. In the depicted embodiment, the flaps 420 are attached to concentric rings 410 that form part of one possible embodiment of a scent cartridge 250 according to the instant invention, the concentric rings 410 have anchors 430 and retainers 440 that hold the flaps 420 in place, permitting their movement when air flow or other forces act upon them. As shown in panel D, the rotation of the device along the path of the depicted arrow causes the flaps 410 to open by the action of centrifugal force. In yet other embodiments, the scent cartridges 250 according to the instant invention have features that prevent the passage of the fragrant or aromatic material out of the openings 400. In one embodiment, the scent cartridges 250 have gas permeable films or filters disposed over the openings 400 that permit air flow, but block the transfer of liquid or solid through the openings 400. Of course one of ordinary skill will appreciate that any one or combinations of these features may be incorporated in the scent cartridges 250 according to the instant invention.

In the various embodiments of the instant invention, the scent dispensing devices 100 include means for driving rotation of the rotor 150. In some embodiments, the drive means is a turbine. As used herein, the term turbine means a rotor assembly that is driven to rotate by the action of a moving fluid, gas, or steam on blades or vanes attached to a hub or plate to produce rotational motion that can be transformed to mechanical power to drive the motion of another object. According to some embodiments of the instant invention, a turbine is used to drive rotational motion of the rotor 150. Referring now to FIG. 4, the depicted embodiment of the scent dispenser 100 includes an assembly as described in connection with FIGS. 1-3, including a rotor 150, housing cover 300, housing base 320, actuation knob 320, and scent cartridge 250. The depicted embodiment also includes a turbine 500, a water-tight seal 600, and fluid inlet 710 and outlet 700 ports. The f fluid inlet 710 and outlet 700 ports allow the flow of fluid through the scent dispenser 100. The inlet port 710 is in operable communication with the turbine 500, such that when the scent dispenser 100 is in use, fluid directed through the inlet port 710 is passed over the turbine 500 to drive its rotation and thereafter passes through the outlet port 700. The turbine 500 is also in operable communication with the rotor 150. In some embodiments, the operable communication between the turbine 500 and the rotor 150 is via a shank, shaft, pin or other coupling that permits the rotational motion of the turbine 500 to drive rotational motion of the rotor 150.

Referring still to FIG. 4, according to the depicted embodiment, the turbine 500 has a central axis X′, a peripheral edge 510, and substantially planar first and second faces 520 and 530. In the depicted embodiment, the rotor is formed of a substantially planar plate. In alternate embodiments, the rotor is formed of a central hub that is either planar or cylindrical in shape. In any of such varied embodiments, the turbine 500 has a plurality of vanes 540 that are radially oriented relative to the center or hub of the turbine 500. In the embodiment depicted in FIG. 4, each vane 540 projects from said first or second face 520 or 530, and has a length that extends from between the peripheral edge 510 of the turbine 500 toward its central axis X′. According to some embodiments, the length of each vane 540 extends from between the peripheral edge 510 and terminates substantially at the central axis X′. According to alternate embodiments, the length of each vane 540 extends from the peripheral edge 510 and terminates at a point before the central axis X. The vanes 540 of the turbine 500 are substantially rectangular in shape, and are either flat or cambered.

The scent dispensers 100 having a fluid driven turbine 500 also have a water-tight seal, such as the plate-shaped seal 600 depicted in FIG. 4. The depicted seal 600 may be of a rigid, semi-rigid, or flexible material, that is fluid impermeable. Attachment or sealing to effectively prevent the passage of fluid from the turbine to the rotor 150 or scent cartridge 250 is achieved using any of a variety of sealing techniques and implements known in the art. For example, the seal 600 may be formed of a self sealing adhesive based or coated material that bonds with the housing to prevent transmission of fluid. Alternatively, the seal 600 may be formed using a gasket (not shown), silicone sealant, or other sealant material. Of course, such a seal may take the form of a component that is integrally molded with a portion of the housing, such as the bottom cover 310.

The turbine 500 drive mechanism of the scent dispenser 100 is operatively connected to an inlet port 710 that is attachable to a fluid source, such as a standard water line or plumbing fixture that is part of a plumbing system. As used herein, the term “plumbing fixture” includes shower and bathtub components, piping systems, inlet pipes and other plumbing components utilized to transfer or outlet water, including cold, warm or hot water, that are generally cylindrical in shape. The term plumbing fixture includes shower heads and the pipes that connect to the shower head outside of the wall enclosing a shower and bathtub spouts and any pipes which connect to the spout outside of the wall enclosing the bath. The term plumbing fixture also includes any specially designed or configured components that can be utilized to carry water and cooperate with the turbine 500 drive system of the scent dispenser 100. One of ordinary skill will appreciate that residential plumbing systems are included among the wide variety of fluid sources and fluid flow systems that are known in the fluid dispensing art. The scent dispensers 100 according to the instant invention that have a fluid driven turbine 500 also have an outlet port 700 for further transmitting the water after its passage over the turbine 500, or after its diversion past the turbine 500.

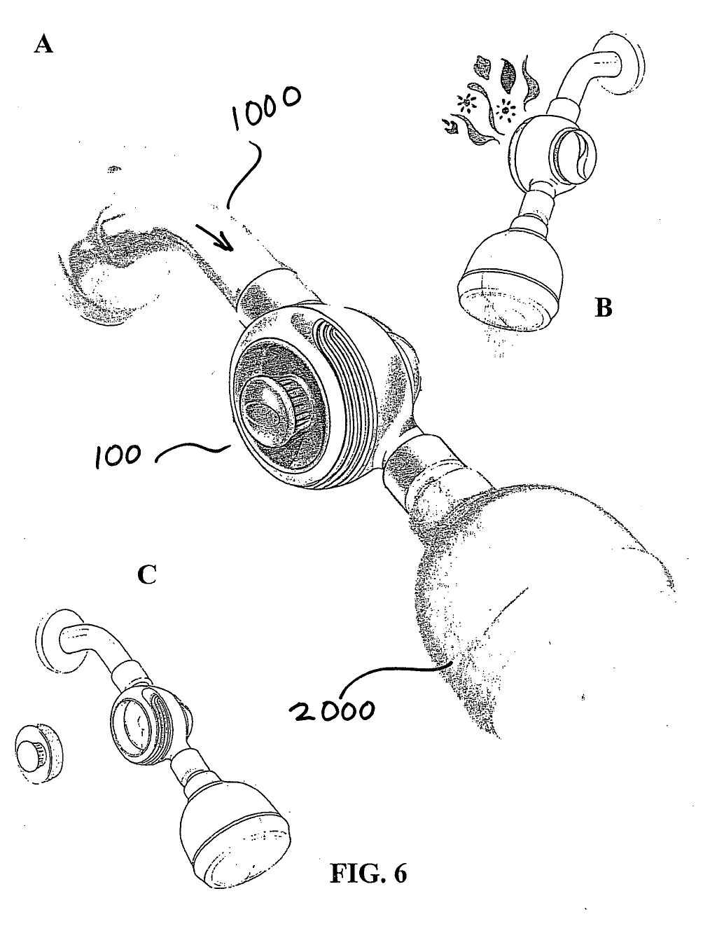

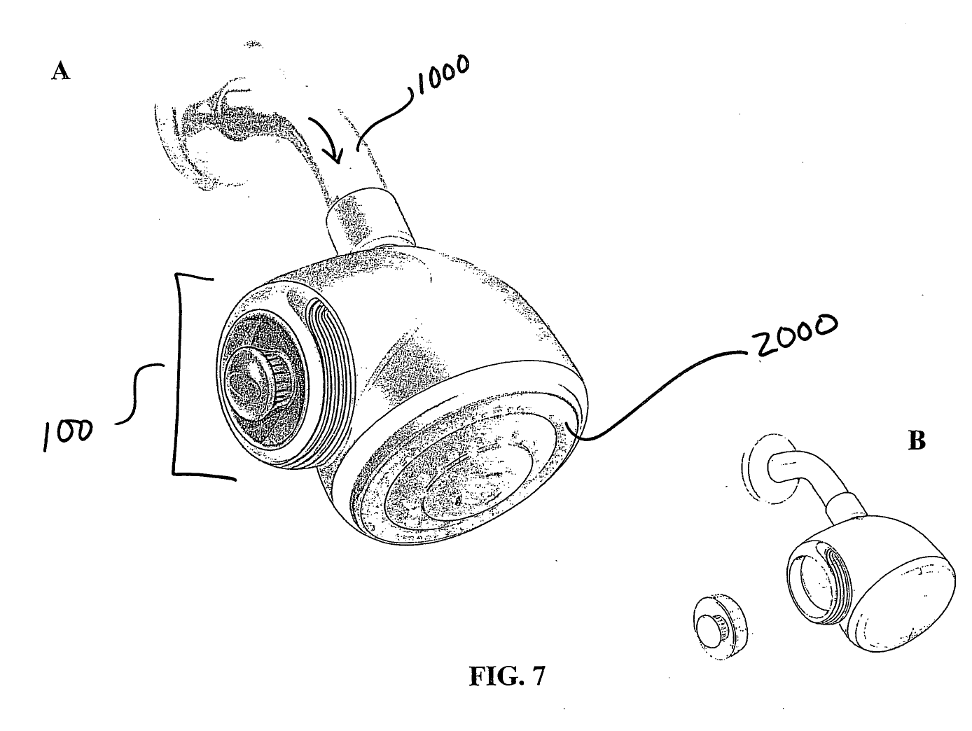

According to some embodiments of the instant invention, and as depicted in FIG. 4 and illustrated in FIG. 6, both the inlet 710 and outlet 700 ports are adapted for attachment in series between a fluid supply 1000 adjacent to the inlet port 710 and a fluid delivery fixture 2000. According to alternate embodiments of the instant invention, and as depicted in FIG. 4 and illustrated in FIG. 7, the inlet port 710 is adapted for attachment to a fluid supply 1000, and the outlet port 700 is a fluid delivery fixture 2000, integral with the scent dispenser 1000. According the various embodiments, a fluid delivery fixture includes devices such as a shower head, sprayer, or jet. In yet other alternate embodiments that are not shown, both the inlet 710 and outlet 700 ports are adapted for attachment in series between a fluid supply 1000 adjacent to the inlet port 710 and a fluid transfer device that further transports the fluid but does not necessarily dispense the fluid from a fluid delivery fixture. For example, the outlet port 700 may deliver the fluid to be recalculated through the scent dispenser 100. According to the various embodiments, two or more scent dispensers 100 may be combined in series or in parallel within a fluid system.

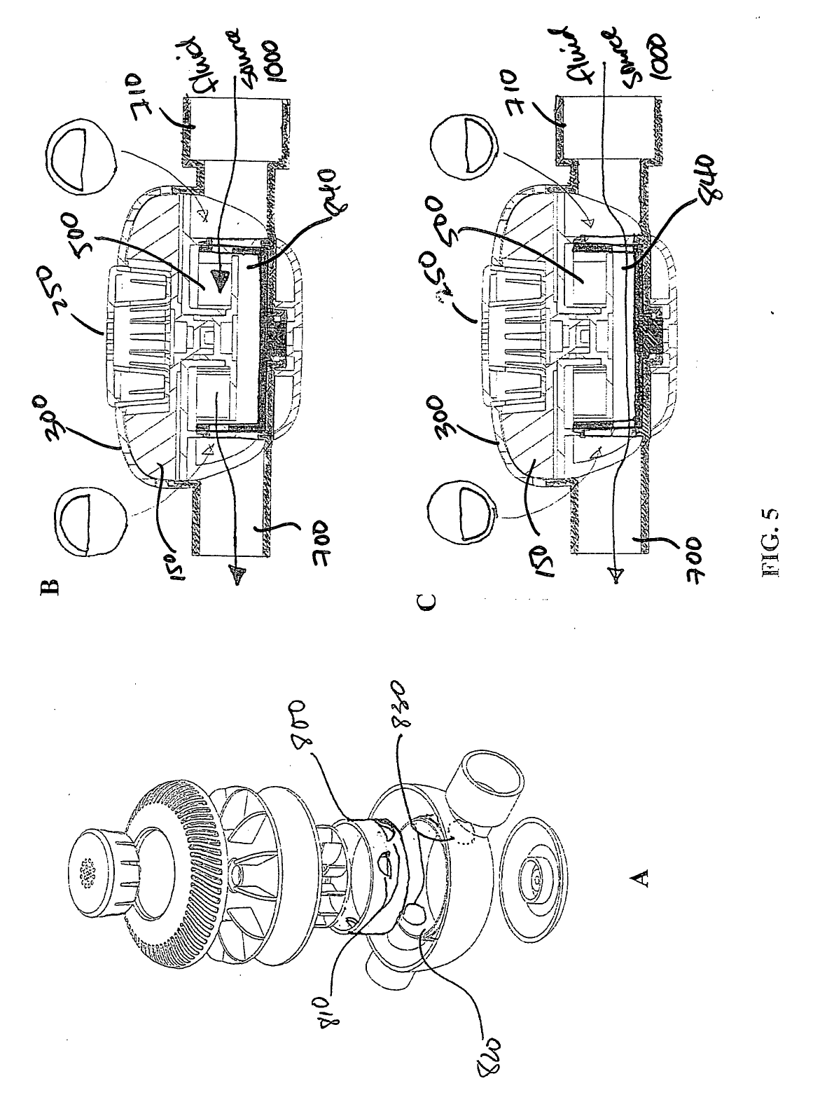

According to some embodiments of the instant invention the scent dispenser 100 has a flow control. As depicted in the embodiment depicted in FIG. 5, and illustrated in FIGS. 6 and 7, the flow control includes an annular ring 800 positioned over the turbine 500, the annular ring 800 having an array of openings 810 in the form of slots or slits around its circumference. The depicted flow control also includes an annular base 820 seated in the housing base 310, the annular base 820 array of openings 830 in the form of slots or slits around its circumference. One of the annular ring 800 and the annular base 820 is fixed in position relative to the components of the turbine 500 drive system. In one embodiment, the annular ring 800 is fixed to the water-tight seal 600; in an alternate embodiment, the annual base 820 is fixed to the housing base 310. The non-fixed annular ring 800 or annual base 820 is operably connected to an actuation means, such as an actuation knob 320, as depicted in FIG. 5. Movement of the actuation means directs movement of the openings of the movable annular ring 800 or annual base 820 either into or out of alignment with the openings of the fixed annular ring 800 or annual base 820. When the openings are aligned, fluid flow over the turbine 500 is permitted, and when the openings are not aligned, fluid flow over the turbine 500 is not permitted, and is diverted through a bypass channel 840. Of course it is understood that there are a variety of alternatives for fixing movement of the annular ring 800 or annual base 820. Likewise, the form of openings in the annular ring 800 or annual base 820 may vary, and include openings having any of a variety of shapes that are circular, ovoid, slit, slot or grid-like. Finally, one of ordinary skill will recognize that the actuation means for controlling a fluid control can be in a variety of forms, including a lever, an internal switch covered by a deformable membrane, and the like.

One of ordinary skill will appreciate that other flow control devices can be used, such as valves or other obstructions positioned within the flow portion of the device, which would serve to block and/or redirect flow. Flow controls including diverter flow channels, alternate outlet ports could be used. Additional methods and devices are known in the art for achieving control of flow in fluid systems and are known in the art, and are included within the scope of the disclosure. As well, the controls for flow into the device could be automatically controlled to turn on or to shut off upon the introduction of fluid flow into the device. For example, the flow control could have spring load or other tension mechanism which is actuated by fluid flow. Still other automated actuation mechanisms are known in the art, and are included within the scope of the disclosure.

In some alternate embodiments of the instant invention, the means for driving the rotor 150 of the scent dispensing devices 100 include is a motor (not shown), more particularly, an electric motor. According to such embodiments, the motor is electrically powered an alternating current adapter for use with standard U.S. household current and a power cord having a plug for insertion into standard household electric receptacle, or a direct current adapter for converting various voltages of direct current to a fixed voltage and a power cord having a plug for insertion into a direct current electric receptacle, or one or more batteries.

Electric motors sufficient to drive rotation of the rotor 150 may rotates an output shaft that is coupled to the rotor 150 at between 12,000 revolutions per minute to 20,000 revolutions per minute when no load is placed on the motor. In some case, the electric motor rotates its output shaft at between 14,000 revolutions per minute to 17,000 revolutions per minute when no load is placed on the motor. Such an electric motor operates on 6 volts. It should be apparent to those skilled in the art, however, that various electric motors could be used, and the present invention is not limited to the disclosed electric motor outputs. In some embodiments, the motor can be driven either by AC or DC power, or by one or more batteries. Good results have been obtained powering motors as describe above using four 1.5 volt AA alkaline batteries (not shown) that can be supported and enclosed within a battery compartment of a scent dispenser (not shown). The batteries can be electronically connected with the electric motor and actuated by an activation switch such as a momentary or other type of switch. Different types and numbers of batteries could be effectively used.

The invention also provides articles of manufacture that include a scent dispenser 100 in a package in association with a set of instructions that direct the consumer how to operate the dispenser 100 and/or the rotor 150 and/or the scent cartridge 250 correctly, to obtain the desirable olfactory, psychological and/or physiological effects. The packaged scent dispenser may be disposable or replenishable, and the scent cartridge 250 will be accordingly removable and replaceable. The articles of manufacture may contain one or more scent cartridges.

The invention also provides methods for dispensing scent into a surrounding area. The methods involve providing, a device having a rotor with a front face, a peripheral edge, a center axis, and having an aromatic or fragrant substance positioned generally at the rotor's center axis. The rotor has vanes that are either parallel to or canted relative to a radial line from front face of the rotor, and the vanes have a shape that is either flat or cambered. The device is operated by driving rotation of the rotor around its axis, and dispenses scent by drawing air over the fragrant substance and expelling the air between the vanes and over the peripheral edge of the rotor. The rotor is driven by a motor or a turbine. The motor is powered by an electrical source. The turbine is powered by the flow of a stream of fluid.

The embodiments described above are examples only, and are not intended to limit the scope of the claims set forth below. Variations to the inventions described herein, including alternate embodiments not specifically described, are quiet possible and are encompassed by the claims as understood by one of ordinary skill in the art. Indeed, the claimed inventions have their broad and ordinary meaning as set forth below in the claims.

1. A scent dispenser for dispensing a scent into a surrounding area comprising:
a rotor defining a cartridge-receiving region and multiple vanes radiating outwardly from the region, the multiple vanes defining multiple spaces therebetween, and
a scent cartridge received in the cartridge-receiving region of the rotor, the scent cartridge containing an aromatic or fragrant substance and having an exterior surface defining multiple openings, at least some of the multiple openings facing the multiple spaces between the vanes so that fragrant substance passing out of the scent cartridge flows into the spaces between the vanes.

2. The scent dispenser according to claim 1, wherein the rotor is adapted to rotate about an axis

3. The scent dispenser according to claim 2, wherein the vanes are arranged radially with respect to this axis of rotation.

4. The scent dispenser according to claim 2, wherein the vanes arranged at acute angles with respect to imaginary radii emanating from this axis of rotation.

5. A scent dispenser according to claim 2, the rotor having a central axis, a peripheral edge and substantially planar first and second faces, at least one face of the rotor comprising the radially oriented plurality of vanes, wherein each vane projects from the face and has a length that extends from between the peripheral edge of the rotor toward its central axis; and
the scent cartridge having an interior space for retaining the aromatic or fragrant substance, an exterior surface, the plurality of openings being generally elliptical, circular, grid, slit or slot shaped.

6. The dispenser according to claim 5, wherein the dispenser, when in use, is assembled with the scent cartridge inserted into the rotor cartridge-receiving region, and the rotor is driven to rotate about its axis, whereby the dispenser is capable of drawing air through the openings in the scent cartridge and passing the air out through the plurality of vanes.

7. The dispenser according to claim 5, comprising a drive means in operative communication with the rotor for driving its rotation.

8. The dispenser according to claim 7, wherein the drive means is a motor.

9. The dispenser according to claim 8, wherein the motor is electrically powered an alternating current adapter for use with standard U.S. household current and a power cord having a plug for insertion into standard household electric receptacle, or a direct current adapter for converting various voltages of direct current to a fixed voltage and a power cord having a plug for insertion into a direct current electric receptacle, or one or more batteries.

10. The dispenser according to claim 7, wherein the drive means is a turbine.

11. The dispenser according to claim 10, the dispenser comprising a fluid inlet port and an outlet port for allowing the flow of fluid through the dispenser, the inlet port in operable communication with the turbine, such that when the dispenser is in use, fluid directed through the inlet port is passed over the turbine to drive its rotation and thereafter passes through the outlet port.

12. The dispenser according to claim 10, the turbine having a central axis, a peripheral edge and substantially planar first and second faces, at least one face of the turbine comprising a plurality of vanes that are radially oriented, each vane projecting from the at least one face and having a length that extends from between the peripheral edge of the turbine toward its central axis.

13. The dispenser according to claim 12, the vanes of the turbine having a shape that is either flat or cambered.

14. The dispenser according to claim 10, comprising a water-tight seal between the rotor and the turbine.

15. The dispenser according to claim 11, comprising a flow control means that can be actuated between an on and off position to control the flow of fluid through the inlet port and across the turbine.

16. The dispenser according to claim 15, comprising a bypass channel for accommodating the flow of fluid when the flow control is in the off position.

17. The dispenser according to claim 11, wherein both the inlet and outlet ports are adapted for attachment in series between a fluid supply adjacent to the inlet port and a fluid delivery fixture.

18. The dispenser according to claim 17, wherein the fluid delivery fixture is a shower head, sprayer, or jet.

19. The dispenser according to claim 11, wherein the inlet port is adapted for attachment to a fluid supply and the outlet port is a fluid delivery fixture.

20. The dispenser according to claim 19, wherein the fluid delivery fixture is a shower head, sprayer, or jet.

21. The dispenser according to claim 1, comprising a housing that encloses the rotor, the housing having an aperture for insertion of the scent cartridge into the cartridge-receiving region of the rotor, and a plurality of openings.

22. The dispenser according to claim 21, the housing being formed of two or more parts.

23. The dispenser according to claim 1, the vanes b having a shape that is flat.

24. The dispenser according to claim 23, the vanes having a shape that is cambered.

25. The dispenser according to claim 24, the vanes having a length that extends from the peripheral edge of the rotor and ends between the peripheral edge of the rotor and its center axis, the cartridge-receiving region for the scent cartridge being formed by ends of the vanes that are between the peripheral edge of the rotor and its center axis, the cartridge-receiving region having a generally circular shape.

26. The dispenser according to claim 1, the rotor comprising a hub or a plate and an aperture therethrough.

27. The dispenser according to claim 1, the scent cartridge having a generally disc-shaped, oval, spherical, hemispherical, parallelepiped-shaped, rectangular, cube-shaped, cuboid-shaped, cylindrical-shaped, pyramid-shaped, spherical-shaped, or irregularly-shaped, the plurality of openings being generally elliptical, circular, grid, slit or slot shaped.

28. The dispenser according to claim 27, the dispenser being replenishable, and the scent cartridge being removable and replaceable.

29. The dispenser according to claim 27, the scent cartridge having a generally disc-shape and having a rotor insertion face, an external face, and a substantially perpendicular edge that is positioned therebetween, wherein the rotor insertion face is contacted with the rotor when the dispenser is assembled.

30. The dispenser according to claim 29, the external face having one or a plurality of openings, and the edge having a plurality of radially spaced openings.

31. The dispenser according to claim 29, the external face having one or a plurality of openings and the rotor insertion face having one or a plurality of openings.

32. The dispenser according to claim 10, the vanes being either parallel to or canted relative to a radial line from the turbine and having a shape that is either flat or cambered.

33. The dispenser according to claim 32, the vanes having a length that extends from between the peripheral edge of the turbine to its center axis.

34. The dispenser according to claim 33, the vanes having a length that extends from between the peripheral edge of the turbine and ends between the peripheral edge of the turbine and its center axis.

35. A scent dispenser for dispensing a scent into a surrounding area comprising:
a fan comprising a cartridge-receiving region that is adapted to receive a scent cartridge; and
a scent cartridge adapted for insertion into the cartridge-receiving region of the fan, the scent cartridge having an interior space for retaining an aromatic or fragrant substance, an exterior surface, and venting to allow the passage of vapor between the exterior of the scent cartridge and its interior space.

36. The dispenser according to claim 35, comprising a vented housing that is adapted with an aperture that aligns with the fan cartridge-receiving region to permit insertion and removal of the scent cartridge.

37. The dispenser according to claim 36, wherein the fan is generally circular and comprises vanes that are either parallel or canted, and the housing is generally dome shaped.

38. The dispenser according to claim 35, the scent cartridge having a generally disc-shaped, oval, spherical, hemispherical, parallelepiped-shaped, rectangular, cube-shaped, cuboid-shaped, cylindrical-shaped, pyramid-shaped, spherical-shaped, or irregularly-shaped, the venting comprising a plurality of openings being generally elliptical, circular, grid, slit or slot shaped.

39. The dispenser according to claim 38, the dispenser being replenishable, and the scent cartridge being removable and replaceable.

40. The dispenser according to claim 35, wherein the dispenser, when in use, is assembled with the scent cartridge inserted into the fan cartridge-receiving region, and the fan is driven to rotate, whereby the dispenser is capable of drawing air through the openings in the scent cartridge and passing the air out through the plurality of fan vanes and the vented housing.

41. The dispenser according to claim 35, comprising a drive means in operative communication with the fan for driving its rotation, wherein the drive means is a motor or a turbine.

42. The dispenser according to claim 41, the drive means comprising a turbine, the dispenser comprising a fluid inlet port and an outlet port for allowing the flow of fluid through the dispenser, the inlet port in operable communication with the turbine, such that when the dispenser is in use, fluid directed through the inlet port is passed over the turbine to drive its rotation and thereafter passes through the outlet port.

43. The dispenser according to claim 42, comprising a water-tight seal between the fan and the turbine.

44. The dispenser according to claim 43, comprising a flow control means that can be actuated between an on and off position to control the flow of fluid through the inlet port and across the turbine and a bypass channel for accommodating the flow of fluid when the flow control is in the off position.

45. The dispenser according to claim 44, wherein both the inlet and outlet ports are adapted for attachment either in series between a fluid supply adjacent to the inlet port and a fluid delivery fixture or wherein the inlet port is adapted for attachment to a fluid supply and the outlet port is a fluid delivery fixture.

46. The dispenser according to claim 45, wherein the fluid delivery fixture is a shower head, sprayer, or jet.

47. The dispenser according to claim 35, wherein the aromatic or fragrant substance is provided in a form selected from the group consisting of: solids, liquids, gels, beads, encapsulates, wicks, a carrier material impregnated with a scent, and combinations thereof.

48. The dispenser according to claim 1, comprising two rotors and two scent cartridges.

49. An article of manufacture comprising the dispenser of claim 1 in a package in association with a set of instructions that direct the consumer how to operate the dispenser and/or the rotor and/or the scent cartridge correctly, to obtain the desirable olfactory, psychological and/or physiological effects.

50. The article of manufacture according to claim 49, the dispenser being replenishable, and the scent cartridge being removable and replaceable, the article of manufacture comprising one or more scent cartridges.

51. A method for dispensing a scent into a surrounding area, comprising the steps of
providing a device comprising a rotor having a front face, a peripheral edge, a center axis, and comprising an aromatic or fragrant substance positioned generally at the rotor's center axis, the rotor comprising vanes that are either parallel to or canted relative to a radial line from the front face of the rotor, the vanes having a shape that is either flat or cambered,
wherein the device is operated by driving rotation of the rotor around its axis, and dispenses scent by drawing air over the fragrant substance and expelling the air between the vanes and over the peripheral edge of the rotor.

52. The method according to claim 51, wherein the method comprises driving the rotation of the rotor with either a motor or a turbine.

53. The method according to claim 52, wherein the rotation of the rotor is driven by a turbine, and the turbine is powered by the flow of a stream of fluid.

54. A replenishable scent dispenser for dispensing a scent into a surrounding area comprising:
a rotor having a central axis, a peripheral edge and substantially planar first and second faces, at least one face of the rotor comprising a plurality of vanes that are radially oriented, each vane projecting from the at least one face and having a length that extends from between the peripheral edge of the rotor toward its central axis, the rotor comprising a cartridge-receiving region at its center that is adapted to receive a scent cartridge, the vanes being either parallel to or canted relative to a radial line from the rotor and having a shape that is either flat or cambered; and
a scent cartridge adapted for removable insertion into the cartridge-receiving region of the rotor and having a generally disc shape comprising a rotor insertion face, an external face, and a substantially perpendicular edge that is positioned therebetween, wherein the rotor insertion face is contacted with the rotor when the dispenser is assembled, the scent cartridge having an interior space for retaining an aromatic or fragrant substance, an exterior surface, and a plurality of openings being generally elliptical, circular, grid, slit or slot shaped to allow the passage of air between the exterior of the scent cartridge and its interior space, the aromatic or fragrant substance provided in a form selected from the group consisting of: solids, liquids, gels, beads, encapsulates, wicks, a carrier material impregnated with ascent, and combinations thereof;
a turbine in operative communication with the rotor for driving its rotation, the turbine having a central axis, a peripheral edge and substantially planar first and second faces, at least one face of the turbine comprising a plurality of vanes that are radially oriented, each vane projecting from the at least one face and having a length that extends from between the peripheral edge of the turbine toward its central axis, the vanes being either parallel to or canted relative to a radial line from the turbine and having a shape that is either flat or cambered; and
a fluid inlet port and an outlet port for allowing the flow of fluid through the dispenser, the inlet port in operable communication with the turbine, such that when the dispenser is in use, fluid directed through the inlet port is passed over the turbine to drive its rotation and thereafter passes through the outlet port, the inlet and outlet ports being adapted for attachment in series between a fluid supply adjacent to the inlet port and a fluid delivery fixture, or the inlet port being adapted for attachment to a fluid supply and the outlet port is a fluid delivery fixture, the fluid delivery fixture is a shower head, sprayer, or jet; and
a water-tight seal between the rotor and the turbine; and
a flow control means that can be actuated between an on and off position to control the flow of fluid through the inlet port and across the turbine, comprising a bypass channel for accommodating the flow of fluid when the flow control is in the off position, and a knob for actuating the flow control means; and
a housing that encloses the rotor, the turbine, and the water-tight seal, the housing having an aperture for insertion of the scent cartridge into the cartridge-receiving region of the rotor, and a plurality of openings;
wherein the dispenser, when in use, is assembled with the scent cartridge inserted into the rotor cartridge-receiving region, and the rotor is driven by the turbine to rotate about its axis, whereby the dispenser is capable of drawing air through the openings in the scent cartridge and passing the air out through the plurality of rotor vanes, thereby emitting scent.

55. A replenishable scent dispenser for dispensing a scent into a surrounding area comprising:
a fan that is generally circular, comprising vanes that are either parallel or canted and a cartridge-receiving region that is adapted to receive a scent cartridge; and
a scent cartridge having a generally spherical, hemispherical or disc shape that is adapted for removable insertion into the cartridge-receiving region of the fan, the scent cartridge having an interior space for retaining an aromatic or fragrant substance, an exterior surface, and venting to allow the passage of air between the exterior of the scent cartridge and its interior space, the venting comprising a plurality of openings being generally elliptical, circular, grid, slit or slot shaped, the aromatic or fragrant substance provided in a form selected from the group consisting of: solids, liquids, gels, beads, encapsulates, wicks, a carrier material impregnated with ascent, and combinations thereof; and
a vented housing that is generally dome shaped and adapted with an aperture that aligns with the fan cartridge-receiving region to permit insertion and removal of the scent cartridge; and
a drive means in operative communication with the fan for driving its rotation, the drive means comprising a turbine, the dispenser comprising a fluid inlet port and an outlet port for allowing the flow of fluid through the dispenser, the inlet port in operable communication with the turbine, such that when the dispenser is in use, fluid directed through the inlet port is passed over the turbine to drive its rotation and thereafter passes through the outlet port, both the inlet and outlet ports being adapted for attachment either in series between a fluid supply adjacent to the inlet port and a fluid delivery fixture, or the inlet port being adapted for attachment to a fluid supply and the outlet port is a fluid delivery fixture, the fluid delivery fixture is a shower head, sprayer, or jet; and
a water-tight seal between the fan and the turbine; and
a flow control means that can be actuated between an on and off position to control the flow of fluid through the inlet port and across the turbine and a bypass channel for accommodating the flow of fluid when the flow control is in the off position; and
wherein the dispenser, when in use, is assembled with the scent cartridge inserted into the fan cartridge-receiving region, and the fan is driven to rotate, whereby the dispenser is capable of drawing air through the openings in the scent cartridge and passing the air out through the plurality of fan vanes.
